(19) United States
(12) Patent Application Publication
(10) Pub. No.: US 2006/0235283 A1
(43) Pub. Date: Oct. 19, 2006

(54) Comfort suite for an intelligent patient bed

(76) Inventors: Valery Vinarov, Palatine, IL (US); Irina Vinarov, Palatine, IL (US)

Correspondence Address: CARDINAL LAW GROUP, Suite 2000, 1603 Orrington Avenue, Evanston, IL, 60201, US

(21) Appl. No.: 11/385,161

(22) Filed: Mar. 21, 2006

Related U.S. Application Data
(60) Provisional application No. 60/663,699, filed on Mar. 21, 2005.

(51) Int. Cl. A61B 5/00; G06Q 10/00; A61B 5/02; A61B 5/04; A61H 1/00

(52) U.S. Cl. 600/300; 600/509; 600/549; 600/485; 705/2; 601/49

(57) ABSTRACT

An intelligent patient bed includes a patient bed equipped with a comfort suite employing a control center in electrical communication with a user interface, a sensor assembly, a comfort control assembly and a virtual patient center for providing comfort to a patient laying on the patient bed as well as an industrious environmental setting for the patient. The control center is also in electrical communication with a care provider center for providing an optimal degree of patient monitoring.

### RELATED APPLICATION DATA

This application claims the benefit of U.S. Provisional Application Ser. No. 60/663,699 filed on Mar. 21, 2005. The entirety of the application is hereby incorporated by reference.

### FIELD OF THE INVENTION

In general, the present invention relates to patients beds designed for the comfort of a patient physically restricted to the bed. More specifically, the present invention relates to a patient bed exhibiting an intelligence for facilitating a maximal degree of comfort for the patient while also facilitating an industrious environmental setting for the patient and an optimal degree of patient monitoring by those taking care of the patient.

### BACKGROUND OF THE INVENTION

Generally, while patient beds have adequately provided comfort to patients, the industry is constantly striving to maximize the degree of comfort for the patient with a view of providing an industrious environmental setting for the patient and an optimal degree of patient monitoring by those taking care of the patient.

In particular, while sleeping, a healthy individual generally turns approximately once every 10-12 minutes. This action provides for healthy blood circulation, stimulation of body organs and movement of body fluids. However, when a person becomes temporarily or permanently immobilized, the blood supply to the parts of the body that is under pressure is restricted. If that pressure is not regularly relieved, and the blood supply restored, the affected tissue dies, sloughs off and a pressure wound, often referred to as ischemic ulcer begins to form. If these ulcers become infected, they can become life threatening.

There are many other medical complications associated with immobility: pneumonia, cardio vascular deficiency, venous stasis, thrombosis, peripheral vascular disease often resulting in amputation, embolism, stone formation, urinary tract infection (kidney and bladder), ESRD, diabetes, muscle wasting, bone demineralization, and atelectasis.

The traditional prevention of pressure ulcers formation method is for a family member, caregiver or institutional employee to regularly turn patient (recommended every two hours) and stabilize the patient in a new position to relieve tissue compression and reestablish blood flow. This has to be done around the clock and, unfortunately, this manual process has a considerable number of drawbacks. Such drawbacks include the waking of the patient every couple of hours during the night, injury to the caregiver during the turning and lifting of the patient, and the cost of providing around the clock care.

Unfortunately, selection of correct prevention methodologies addressing immobility patients, especially in early stages, is often effected by cost factors, such as, the costs for rotating the patient and for the therapeutic bed.

Unfortunately, as mentioned above, pressure ulcers are not the only problem faced by patients effected by immobility. Moreover, quite often, immobility is only one of the characteristic syndromes for many illnesses. Immobility affect patients suffering from spinal cord injuries (with or without ventilator), muscular dystrophy and multiple sclerosis, severe head injuries and stroke, severe pulmonary disease, aging, cancer, Guillain Barre Syndrome, AIDS, coma, amputations, osteomyelitis, degenerative disk disease, post-spinal fusion and laminectomy, rheumatoid arthritis, ALS—amyotrophic lateral sclerosis and any other condition where turning assistance is required.

Pressure ulcers are of major concern to the sufferers, their caregivers and the medical community. The scale of the problem is immense. It is estimated that pressure ulcers effect 1.2 million people each year in the United States alone and that the cost to heal complex pressure ulcers may run as much as $70,000. Most recently, the US Centers for Medicare and Medicaid (formerly Health Care Financing Administration) included pressure ulcers as one of three sentinel events for long term care; formation or subsequent deterioration of pressure ulcer can lead to significant monetary penalties—up to $10,000 per day in long-term care. The current cost to the US Health Care System to treat these and other associated conditions is estimated at $15-$40 billion annually. It is further reported that there are 60,000 deaths annually from complications arising from bedsores.

It is desirable, therefore, to provide a system and method for preventing the formation of pressure ulcers that overcomes these and other disadvantages.

### SUMMARY OF THE INVENTION

One form of the present invention is an intelligent patient bed comprising a patient bed and a comfort suite equipped to the patient bed, wherein the comfort suite includes comfort means for facilitating a maximal degree of comfort for a patient of the intelligent patient bed, an industrious environmental setting for the patient, and an optimal degree of patient monitoring by a care provider of the patient.

A second form of the present invention is an intelligent patient bed comprising a patient bed and a comfort suite equipped to the patient bed, wherein the comfort suite is operably coupled to local device(s) and remote device(s) for facilitating a maximal degree of comfort for a patient of the intelligent patient bed, an industrious environmental setting for the patient, and an optimal degree of patient monitoring by a care provider of the patient.

The aforementioned forms and other forms as well as objects and advantages of the present invention will become further apparent from the following detailed description of various embodiments of the present invention read in conjunction with the accompanying drawings. The detailed description and drawings of the various embodiments of the present invention are merely illustrative of the present invention rather than limiting, the scope of the present invention being defined by the appended claims and equivalents thereof.

### BRIEF DESCRIPTION OF THE DRAWINGS

FIG. 1 illustrates an intelligent patient bed in accordance with the present invention;

FIGS. 2A-2D illustrate various patient beds known in the art;

FIG. 3 illustrates one embodiment in accordance with the present invention of the comfort suite illustrated in FIG. 1;

FIG. 4 illustrates one embodiment in accordance with the present invention of the sensor assembly illustrated in FIG. 3;

FIG. 5 illustrates one embodiment in accordance with the present invention of the comfort control assembly illustrated in FIG. 3;

FIG. 6 illustrates one embodiment in accordance with the present invention of the virtual patient center illustrated in FIG. 3;

FIG. 7 illustrates one embodiment in accordance with the present invention of the care provider center illustrated in FIG. 3;

FIG. 8 illustrates an exemplary array of local devices operatively connected to the comfort suite illustrated in FIG. 1; and

FIG. 9 illustrates an exemplary array of remote devices operatively connected to the comfort suite illustrated in FIG. 1.

### DETAILED DESCRIPTION OF THE PRESENT INVENTION

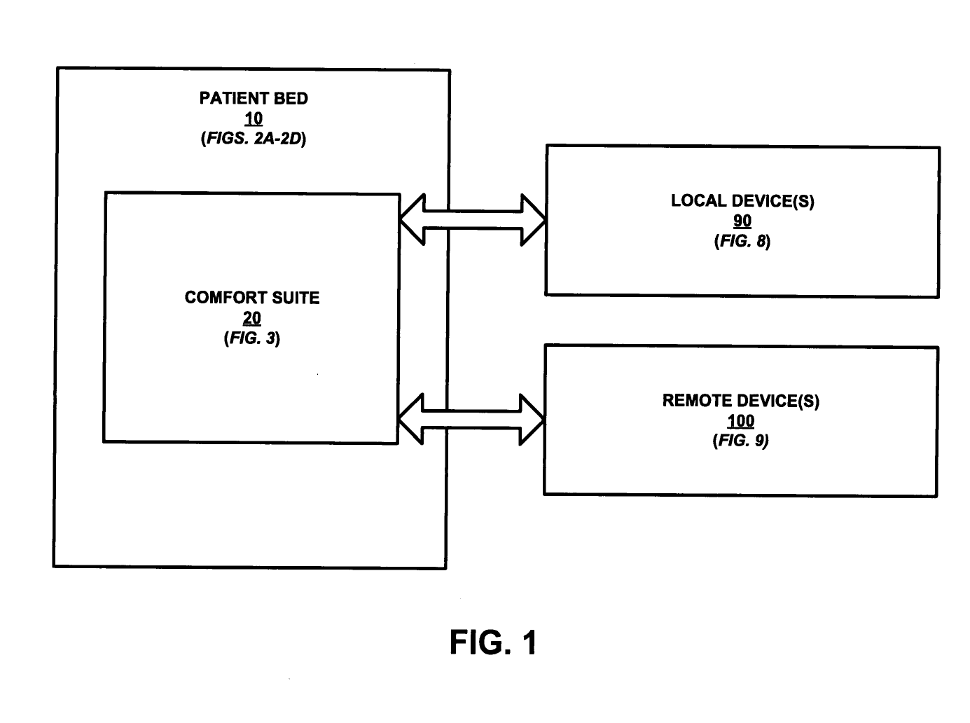

FIG. 1 illustrates an intelligent patient bed of the present invention. Commercially, various embodiments of the intelligent bed of the present invention are intended to be sold under the trade name Phoenix Comfort Suite™.

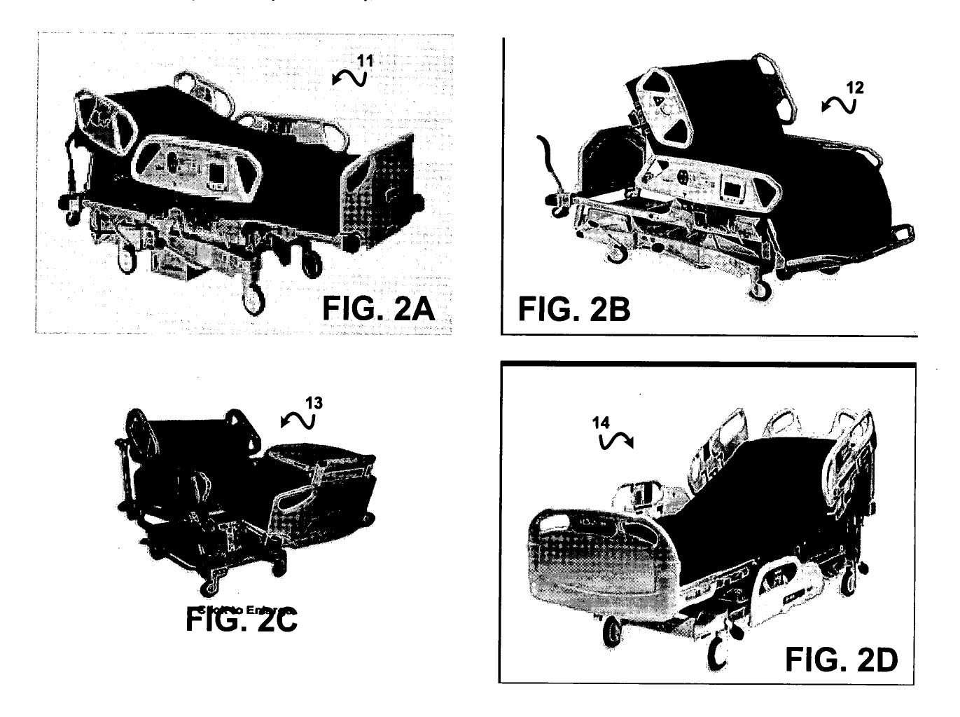

Referring to FIG. 1, the intelligent patient bed employs a patient bed 10 equipped with a comfort suite 20 for facilitating a maximal degree of comfort for a patient physically restricted to patient bed 10 while also facilitating an industrious environmental setting for the patient and an optimal degree of patient monitoring by those taking care of the patient. For purposes of the present invention, the term “patient bed” is broadly defined herein as any type of furniture on or in which a person can lie and sleep, such as, for example, the various beds 11-14 illustrated in FIGS. 2A-2D, respectively. Also for purposes of the present invention, the term “patient” is broadly defined herein as an individual awaiting or under medical care and treatment, and the term “physically restricted” is broadly defined herein as a temporary or permanent confinement of a patient on or in the patient bed.

In practice, the techniques used to equip a patent bed (e.g., patient beds 11-14 shown in FIGS. 2A-2D) with a comfort suit in accordance with the present invention is without limit.

Still referring to FIG. 1, comfort suite 20 facilitates a maximal degree of comfort for a patient physically restricted to patient bed 10 by intelligently implementing one or more systems, devices and/or apparatuses designed to adjust the physical layout of the patient on or in patient bed 10, and by intelligently implementing one or more systems, devices and/or apparatuses designed to provide various therapies as the patient is on or in patient bed 10. Examples of such therapies include, but are not limited to, a continuous lateral motion therapy, a wave comfort therapy, a percussion therapy, and a vibration therapy.

Comfort suite 20 facilitates an industrious environmental setting for the patient by intelligently implementing one or more systems, devices and/or apparatuses designed to establish a quality of life for a patient that enables the patient to learn, work and/or be entertained while being physically restricted to patient bed 10. Examples include, but are not limited to, computers modeled around qualities and standards that are important for the patient while supporting increased flexibility and real-time responsiveness based on state-of-the-art features and technology.

Comfort suite 20 facilitates optimal degree of patient monitoring by those taking care of the patient by intelligently implementing an interface for responsively providing patient information needed by the care provider. Examples include, but are not limited to, interfaces for collecting data related to physical attributes of the patient (e.g., blood pressure, heart rate, etc.) and interfaces for monitoring the degree to which various therapies are being applied to the patient.

Comfort suite 20 accomplishes the aforementioned functions by employing a control center for centralized and controlled management of the various systems, devices and/or apparatuses installed on patient bed 10. For purposes of the present invention, the term “control center” is broadly defined herein as a physical arrangement in a computing environment that contains the basic circuitry and components including, but not limited to, one or more microprocessors, memory, BIOS, expansion slots, and interconnecting circuitry. Additional I/O controllers, peripheral devices/components can be added to the control center through expansion slots and high speed interfaces (e.g., USB 2.0 and IEEE 1394).

In practice, the techniques by which the control center of comfort suite 20 centralizes a control and management of various systems, devices and/or apparatuses installed on patient bed 10 is without limit. In one embodiment, commercially available patient beds (e.g., beds 11-14 shown in FIGS. 2A-2D) can be equipped in accordance with the present invention with the control center of comfort suite 20 structurally designed to control various systems, devices and/or apparatuses for facilitating a maximal degree of comfort for a patient physically restricted to patient bed 10 while also facilitating an industrious environmental setting for the patient and an optimal degree of patient monitoring by those taking care of the patient. Alternatively, a patient bed having a novel and unique structural frame can be equipped in accordance with the present invention with the control center of comfort suite 20 structurally designed to control various systems, devices and/or apparatuses for facilitating a maximal degree of comfort for a patient physically restricted to patient bed 10 while also facilitating an industrious environmental setting for the patient and an optimal degree of patient monitoring by those taking care of the patient.

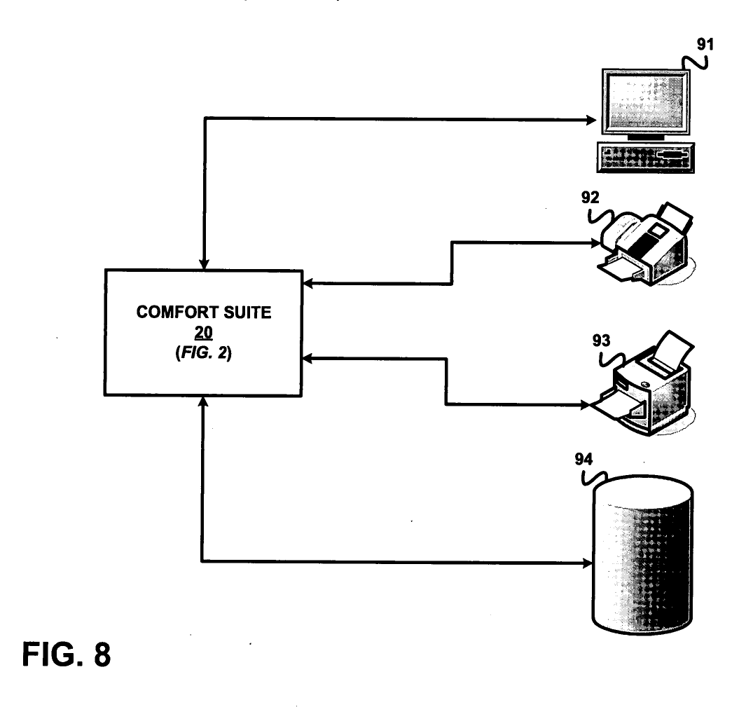

Still referring to FIG. 1, for purposes of supporting the aforementioned various functions, the control center of comfort suite 20 can be in electrical communication with up to X number of local devices 90, where X≧1, and can be in electrical communication up to Y number of remote devices 100, where Y≧1. For purposes of the present invention, the term “local device” is broadly defined herein as a piece of equipment or a mechanism designed to serve a special purpose or to perform a special function that is physically accessible by a care provider while in the presence of the patient bed. As illustrated in FIG. 8, examples of such local devices include, but are not limited to: (1) a virtual and logical workstation 91, (2) a printer 92; (3) a fax machine 93, and (4) a database 94. In one embodiment, local devices 91-94 are physically allocated as part of the control center and designed to enable an impaired patient to learn, work and/or be entertained as well as support special needs of blind, deaf and voiceless patients.

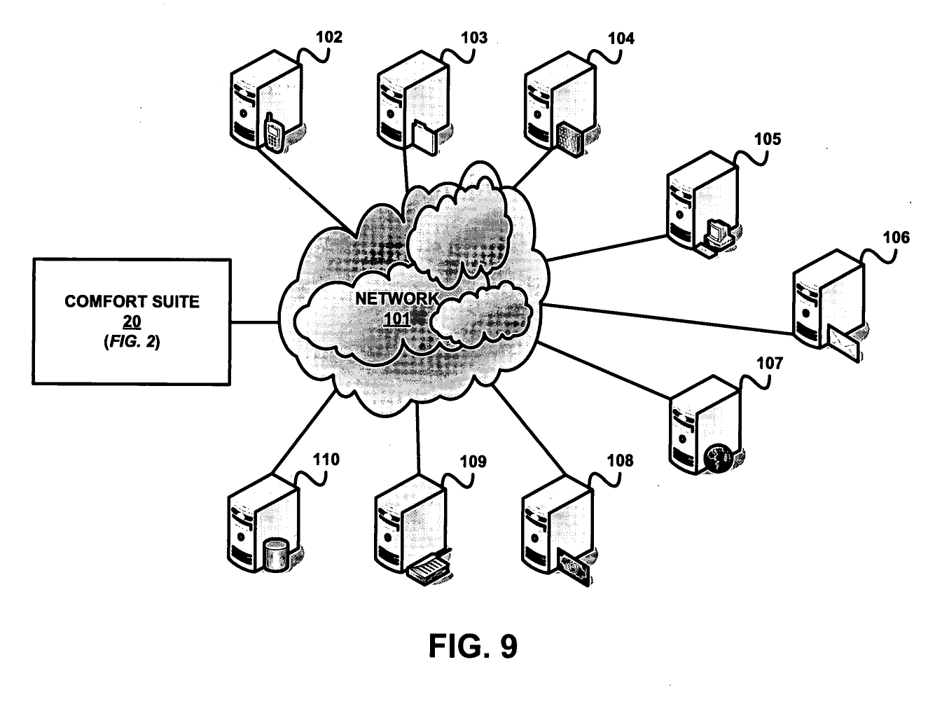

For purposes of the present invention, the term “remote device” is broadly defined herein as a pieces of equipment and/or a mechanism designed to serve a special purpose or to perform a special function of a core network supporting business infrastructure of a care provider facilities and that previously was not physically accessible by a care provider while in the presence of the patient bed. As illustrated in FIG. 9, examples of such remote devices include, but are not limited to: (1) a mobile communication server 102; (2) a file server 103; (3) a proxy server 104; (4) a management server 105; (5) an email server 106; (6) a web server 107; (7) an e-commerce server 108; (8) a directory server 109; and (9) a database server 110.

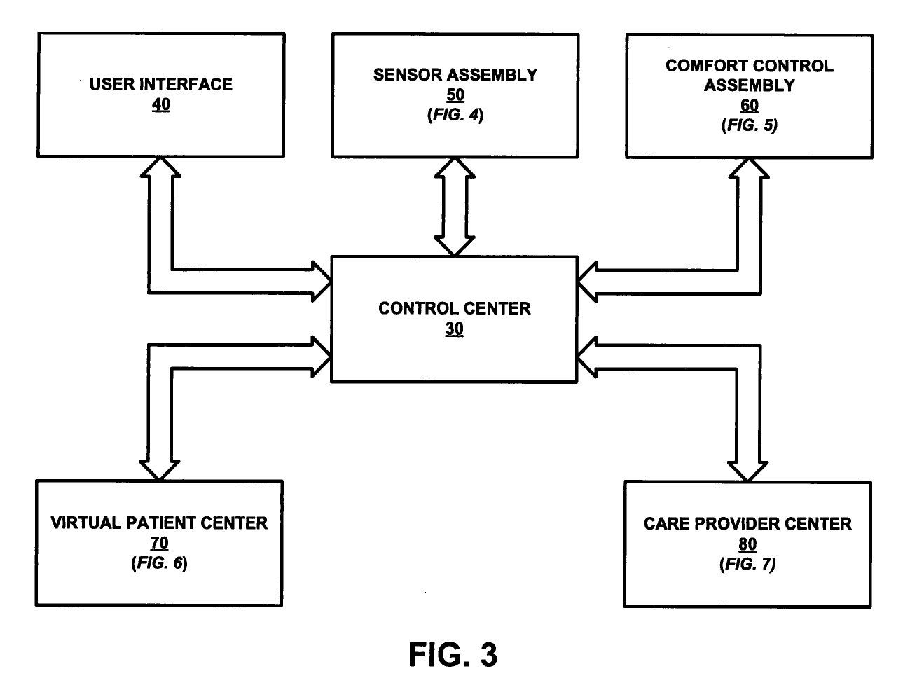

FIG. 3 illustrates one embodiment of comfort suite 20 employing a control center 30. Referring to FIG. 3, control center 30 is in electrical communication with a user interface 40, a sensor assembly 50, a comfort control assembly 60 and a virtual patient center 70 whereby control center 30 utilizes various signals from user interface 40 and sensor assembly 50 as well as information provided by virtual patient center 70 to operate comfort control assembly 60 in a manner that provides a maximum degree of comfort to a patient lying on an intelligent patient bed of the present invention. Virtual patient center 70 is also utilized to facilitating an industrious environmental setting for the patient.

In one embodiment, control center 30 is an Intel Processor based motherboard having Windows XP as its operating system, and an array of I/O controllers designed to implement various principles of the present invention. User interface 40 includes any number of graphical user interfaces and/or remote controls.

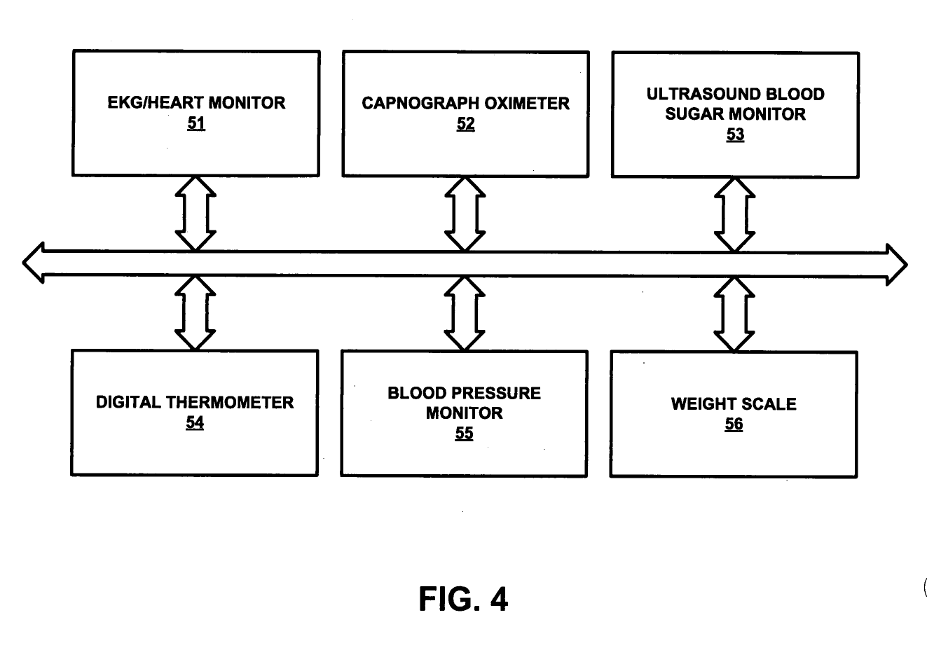

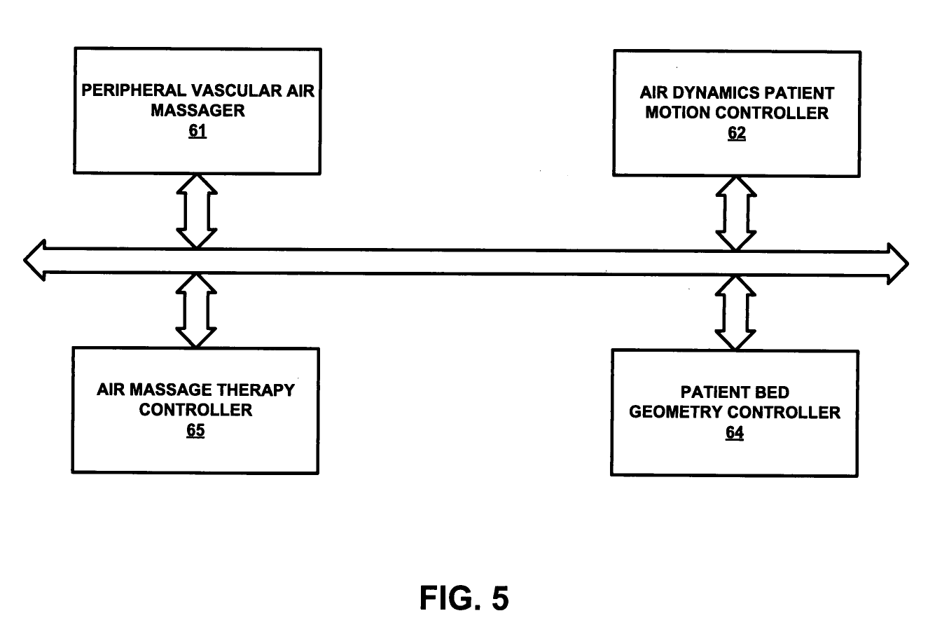

Sensor assembly 50 includes a EKG/heart monitor 51, a capnograph oximeter 52, a ultrasound blood sugar monitor 53, a digital thermometer 54, a blood pressure monitor 55, and a weight scale 56 as illustrated in FIG. 4. Comfort control assembly 60 includes a peripheral vascular air massager 61, an air dynamics based patient motion controller 62, an air massage therapy controller 63, and a patient bed geometry controller 64 as illustrated in FIG. 5.

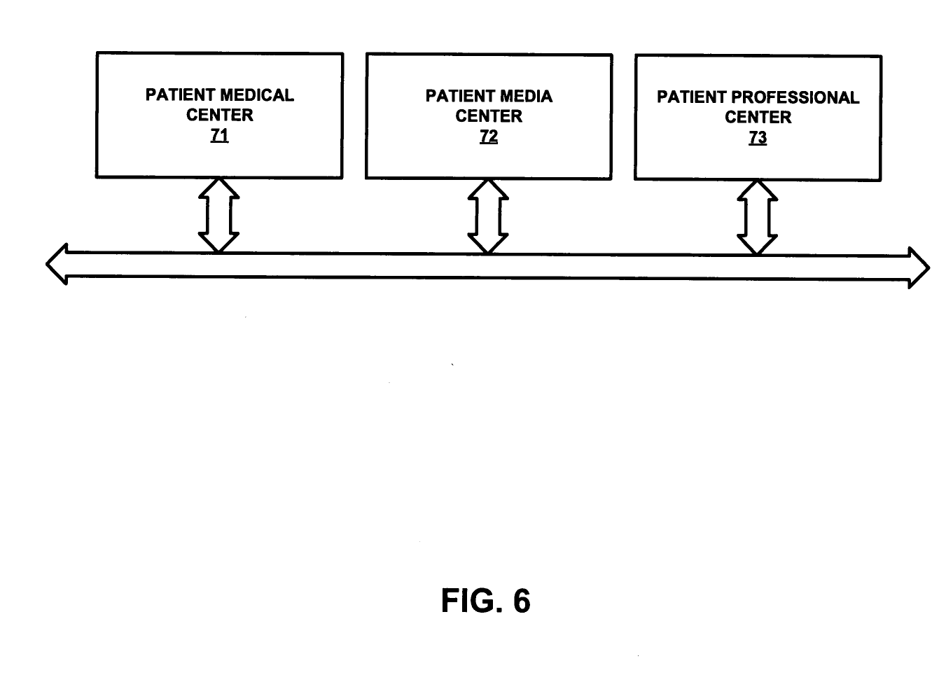

Center 70 includes a patient medical center 71 for facilitating an involvement of the patient with his/her treatment(s) and monitored physical attributes, a patient medical center 72 for entertaining the patient and a patient professional center 73 for allowing the patient to work from the patient bed as illustrated in FIG. 6. In practice, centers 71-73 may or may not be implemented with the same hardware equipped to patient bed 10.

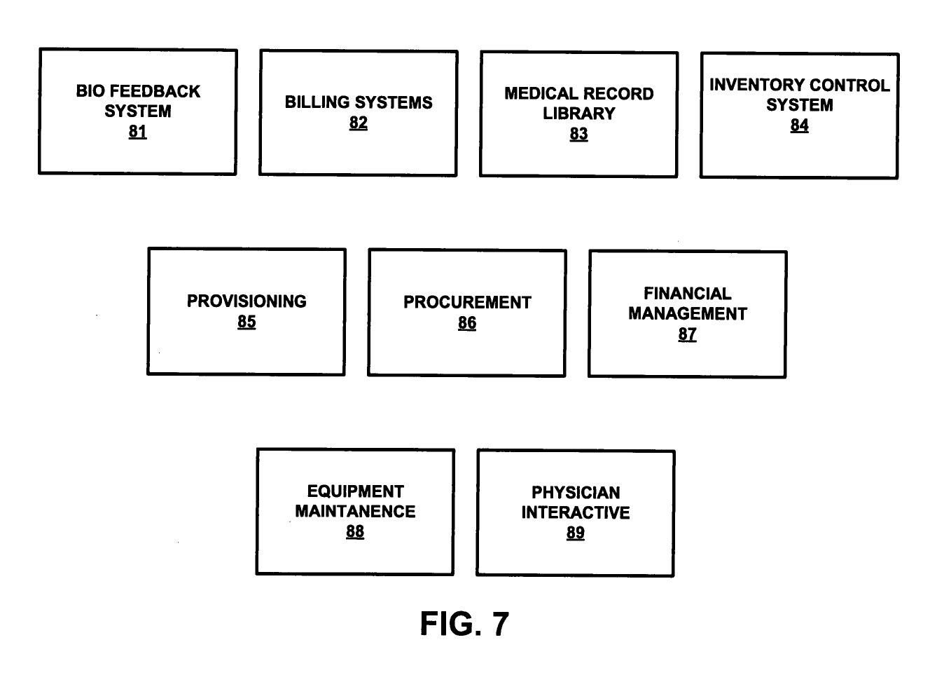

Control center 30 is in further electrical communication with a care provider center 80 for facilitating an optimal degree of patient monitoring. Center 80 includes a bio feedback system 81, a billing system 82, a medical record library 83, an inventory controller 84, a provisioning controller 85, a procurement controller 86, a financial manager 87, an equipment maintenance engine 88, and a physician interactive device 89 (e.g., a personal data assistant) as illustrated in FIG. 7. As such, control center 30 will establish fundamentally new communication paradigm in the medical industry.

Specifically, information relayed to care provider 80 is sent in response to the treatment and procedures that the patient undergoes and may be sent automatically from control center 30 in response to information input by the medical care provider. The information may also be sent automatically in response to the completion of a test performed on the patient. For example, the patient may be connected to blood sugar monitor 53 (FIG. 4) for monitoring the blood sugar level of the patient without the assistance of the care provider. When the test is completed, the results may be communicated to billing systems 82, medical records library 83, and inventory controller 84.

In summary, medical treatment and care will be revolutionized by the convergence of new technologies, integrated operations support, real-time applications and high-speed wireless networking. Control center 30 supports directed interface and data exchange with institution core network (e.g., remote devices 102-109 shown in FIG. 9) to thereby trigger medical procedure activity in real-time based on applications installed on the network (e.g., applications 81-89 shown in FIG. 7 installed on remote devices 102-109 shown in FIG. 9).

From the description herein of FIGS. 1-9, one skilled in the art will appreciate the many benefits of a patient bed constructed and operated in accordance with the present invention. The present invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

We claim:

1. An intelligent patient bed, comprising:
a patient bed; and
a comfort suite equipped to the patient bed, wherein the comfort suite includes comfort means for facilitating a maximal degree of comfort for a patient of the intelligent patient bed, an industrious environmental setting for the patient, and an optimal degree of patient monitoring by a care provider of the patient.

2. The intelligent patient bed of claim 1, wherein the comfort means includes:
a control center in electrical communication with a user interface for providing patient information needed by the care provider.

3. The intelligent patient bed of claim 1, wherein the comfort means includes:
a control center in electrical communication with a sensor assembly for collecting data related to at least one physical attribute of the patient.

4. The intelligent patient bed of claim 3, wherein the sensor assembly includes at least one of a EKG/heart monitor, a capnograph oximeter, an ultrasound blood sugar monitor, a digital thermometer, a blood pressure monitor and a weight scale.

5. The intelligent patient bed of claim 1, wherein the comfort means includes:
a control center in electrical communication with a comfort control assembly for providing at least one therapy to the patient.

6. The intelligent patient bed of claim 5, wherein the comfort control assembly includes at least one of a peripheral vascular air massager, an air dynamics patient motion controller, an air massage therapy controller, and a patient bed geometry controller.

7. The intelligent patient bed of claim 1, wherein the comfort means includes:
a control center in electrical communication with a virtual patient center for providing the industrious environmental setting for the patient.

8. The intelligent patient bed of claim 7, wherein the virtual patient center includes at least one of a patient medical center for facilitating an involvement of the patient with the treatment and monitored physical attributes, a patient medical center for entertaining the patient and a patient professional center for allowing the patient to work from the patient bed.

9. The intelligent patient bed of claim 1, wherein the comfort means includes:
a control center in electrical communication with a care provider center for providing the optimal degree of patient monitoring by the care provider of the patient.

10. The intelligent patient bed of claim 9, wherein the care provider center includes at least one of a bio feedback system, a billing system, a medical record library, an inventory control system, a provisioning system, a procurement system, a financial management system, an equipment maintenance system and a physician interactive device.

11. An intelligent patient bed, comprising:
a patient bed; and
a comfort suite equipped to the patient bed, wherein the comfort suite is operably coupled to at least one local device and at least one remote device for facilitating maximal degree of comfort for a patient of the intelligent patient bed, an industrious environmental setting for the patient, and an optimal degree of patient monitoring by a care provider of the patient.

12. The intelligent patient bed of claim 11, wherein the comfort suite includes:
a control center in electrical communication with a user interface for providing patient information needed by the care provider.

13. The intelligent patient bed of claim 11, wherein the comfort suite includes:
a control center in electrical communication with a sensor assembly for collecting data related to at least one physical attribute of the patient.

14. The intelligent patient bed of claim 13, wherein the sensor assembly includes at least one of a EKG/heart monitor, a capnograph oximeter, an ultrasound blood sugar monitor, a digital thermometer, a blood pressure monitor and a weight scale.

15. The intelligent patient bed of claim 1, wherein the comfort suite includes:
a control center in electrical communication with a comfort control assembly for providing at least one therapy to the patient.

16. The intelligent patient bed of claim 5, wherein the comfort control assembly includes at least one of a peripheral vascular air massager, an air dynamics patient motion controller, an air massage therapy controller, and a patient bed geometry controller.

17. The intelligent patient bed of claim 1, wherein the comfort suite includes:
a control center in electrical communication with a virtual patient center for providing the industrious environmental setting for the patient.

18. The intelligent patient bed of claim 17, wherein the virtual patient center includes at least one of a patient medical center for facilitating an involvement of the patient with the treatment and monitored physical attributes, a patient medical center for entertaining the patient and a patient professional center for allowing the patient to work from the patient bed.

19. The intelligent patient bed of claim 11, wherein the comfort suite includes:
a control center in electrical communication with a care provider center for providing the optimal degree of patient monitoring by the care provider of the patient.

20. The intelligent patient bed of claim 19, wherein the care provider center includes at least one of a bio feedback system, a billing system, a medical record library, an inventory control system, a provisioning system, a procurement system, a financial management system, an equipment maintenance system and a physician interactive device.
